(19) United States
(12) Patent Application Publication
(10) Pub. No.: US 2019/0110854 A1
(43) Pub. Date: Apr. 18, 2019

(54) Method and apparatus for frame-based stereotaxy

(71) Applicants: Ashok PILLAI, Miami, FL (US); Akshay NAGARAJAN, Amritapure (IN)

(72) Inventors: Ashok PILLAI, Miami, FL (US); Akshay NAGARAJAN, Amritapure (IN)

(73) Assignee: Turiya Medical LLC, Miami, FL (US)

(21) Appl. No.: 15/932,872

(22) Filed: May 11, 2018

Related U.S. Application Data
(60) Provisional application No. 62/505,103, filed on May 11, 2017.

(51) Int. Cl. A61B 90/11

(52) U.S. Cl. CPC A61B 90/11; A61B 2090/103

(57) ABSTRACT

Exemplary embodiments of the present disclosure disclose a device and methodology for facilitating rapid stereotactic guidance for placing multiple trajectories lateral to a patient's head. The device assembly comprises an adapter, which when fixed to a ring of a stereotactic arc, allows trajectory entry and targeting orthogonal to the sagittal plane using only the y and z frame coordinates. The x coordinate represents the insertion depth in the axial and coronal planes. This adapter is herein referred to as the Z-post adapter and comprises an adaptive collar, a central locking plug, instrument guide inserts of variable lengths and diameters, and an external collar fastener. In alternate embodiments the Z-post adapter can be mounted on an adjustable gimbal mechanism to facilitate oblique trajectories as well. The collar, fastener and gimbal mechanism can be adapted to suit conventional stereotactic frames.

### CROSS-REFERENCE TO RELATED APPLICATION

This application claims the benefit of U.S. Provisional Application No. 62/505,103, filed May 11, 2017, which is hereby incorporated by reference in its entirety

### TECHNICAL FIELD

The present disclosure relates to medical devices and procedures and, more particularly, to frame-based stereotaxy and equipment therefor.

### BACKGROUND

Stereotactic surgery, or stereotaxy, is a form of surgical intervention that utilizes a three-dimensional coordinate system to locate targets within the body in order to perform some surgical action, such as ablation, injection, biopsy, etc. Stereotaxy utilizes geometric information obtained from medical images, such as computed tomography (CT) or magnetic resonance imaging (MRI), to register a set of image data to a subject for surgical guidance. To establish a trajectory in alignment with the registered data, a frame system is applied, though expensive robotic frameless systems are now available. Frame-based stereotaxy has the advantage of providing maximal accuracy in implantation, albeit at the expense of increased operative and planning time

Frame systems can be orthogonal or arc-based. Orthogonal stereotaxy utilizes a frame structure developed by prominent neurosurgeon, Jean Talairach, and has popular application in Stereoelectroencephalography (SEEG) electrode implantation and brain mapping. The Talairach frame was specifically designed to target deeper structures while simultaneously traversing the cerebral cortex using trajectories orthogonal to the vertical plane of the anterior commissure-posterior commissure (AC-PC).

Almost contemporary to Talairach's space and mapping developments, Lars Leksell introduced the concept of arc-based stereotaxy in which the center of the arc was assigned to x, y, and z coordinates of his stereotaxy frame. The arc-centered principle tends to maximize surgical precision at the target, irrespective of the surgical trajectory, and provides a great degree of surgical precision. For this reason arc-based stereotaxy remains the most popular technique used by stereotactic neurosurgeons in the present era. Common procedures include stereotactic biopsies, electrode placement for deep brain stimulation (DBS), or SEEG. When performing SEEG electrode implantations, however, multiple orthogonal trajectories are preferred for several reasons. Thus, many experienced SEEG surgeons prefer SEEG implantation schemes utilizing predominantly orthogonal lateral entry trajectories combined with occasional oblique trajectories. Present options for orthogonal planning of multiple stereotactic trajectories include frame-based, frameless navigation, and robotic techniques.

Many stereotactic neurosurgeons today are accustomed to either the Leksell® or CRW® stereotaxy frames, which are arc-based. Performing multiple orthogonal trajectories using these arc-based systems typically requires cumbersome placement of the arc in the sagittal plane, which is time consuming. With the resurgence of Stereo-EEG in epilepsy surgery centers across the world, and the prevalence of arc-based frames, what is needed is a cost-efficient and convenient method that allows for a multiplicity of orthogonal trajectories with the least amount of stereotactic frame manipulations.

### SUMMARY

The present disclosure relates to a device and methodology for facilitating rapid stereotactic guidance for placing multiple trajectories lateral to a patient's head. The device assembly comprises an adapter, which when fixed to a ring of a stereotactic arc, allows trajectory entry and targeting orthogonal to the sagittal plane using only the y and z frame coordinates. The x coordinate represents the insertion depth in the axial and coronal planes. This adapter is herein referred to as the Z-post adapter and comprises an adaptive collar, a central locking plug, instrument guide inserts of variable lengths and diameters, and an external collar fastener. The Z-post adapter allows stereotactic lateral entry to the head orthogonal to the sagittal plane of the frame, utilizing arc-based frame equipment, thereby obviating the need to use of an arc-based technique for multiple lateral entry trajectories. In alternate embodiments the Z-post adapter can be mounted on an adjustable gimbal mechanism to facilitate oblique trajectories as well. The collar, fastener and gimbal mechanism can be adapted to suit conventional stereotactic frames.

Other objects, features, and advantages will be apparent to persons of ordinary skill in the art from the following detailed description and the accompanying drawings.

### BRIEF DESCRIPTION OF THE DRAWINGS

The above and/or other exemplary features and advantages of the preferred embodiments of the present disclosure will become more apparent through the detailed description of exemplary embodiments thereof with reference to the accompanying drawings, in which:

FIG. 1 depicts an exploded of a Z-post adapter in accordance with an embodiment of the present disclosure;

FIG. 2 depicts an assembly of a Z-post adapter with a Gimbal mechanism in accordance with an embodiment of the present disclosure;

FIG. 3A depicts a cross-sectional view of an assembled Z-post adapter in accordance with an embodiment of the present disclosure;

FIG. 3B depicts an elevation view of an assembled Z-post adapter in accordance with an embodiment of the present disclosure;

FIG. 3C depicts a plan view of an assembled Z-post adapter in accordance with an embodiment of the present disclosure;

FIG. 4 depicts an exploded of a Z-post adapter with a stereotactic frame in accordance with an embodiment of the present disclosure; and

FIG. 5 is a flowchart presenting a use methodology of a Z-post adapter in accordance with an embodiment of the present disclosure.

Throughout the drawings, like reference numbers and labels should be understood to refer to like elements, features, and structures.

### DETAILED DESCRIPTION

Exemplary embodiments of the present disclosure will now be described more fully with reference to the accompanying drawings. The matters exemplified in this description are provided to assist in a comprehensive understanding of various embodiments disclosed with reference to the accompanying figures. Accordingly, those of ordinary skill in the art will recognize that various changes and modifications of the embodiments described herein can be made without departing from the scope and spirit of the claimed inventions. Descriptions of well-known functions and constructions are omitted for clarity and conciseness. To aid in clarity of description, the terms “upper,” “lower,” “above,” “below,” “left” and “right,” as used herein, provide reference with respect to orientation of the accompanying drawings and are not intended to be limiting.

The present disclosure presents an apparatus and method to facilitate guidance of surgical instruments using stereotaxy. Stereotaxy is a method for locating a target within a three-dimensional object, and is used in the medical arts and sciences to locate a target in the human body, in particular in the brain or spine, for medical and surgical treatment.

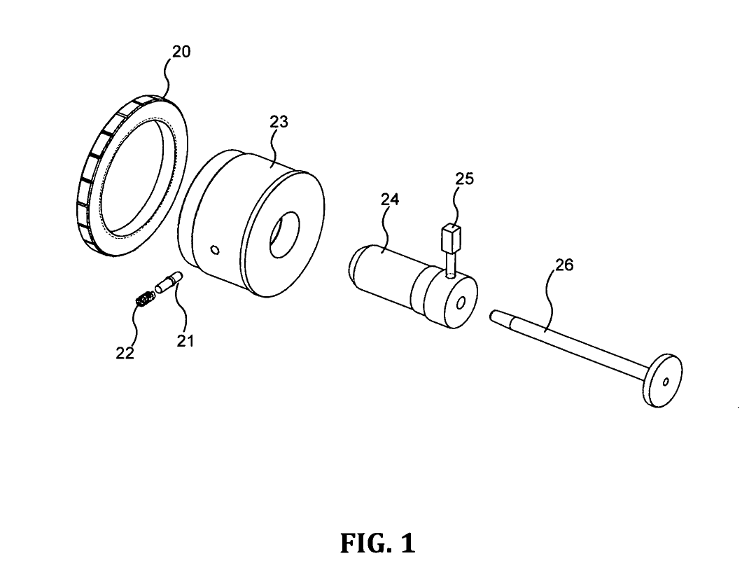

Referring now to FIGS. 1-4, exemplary embodiments of the Z-post adapter are described in accordance with exemplary embodiments of the present disclosure. With respect to FIG. 1, Embodiments of the Z-post adapter comprise a collar 23 that can be releasably mounted directly on a stereotactic frame using a fastener 20, in the case of accessing orthogonal trajectories, or alternatively using a gimbal mechanism ring 11 and fastener 10 for accessing oblique trajectories. The collar 23 forms a central access aperture within which a locking plug 24 is releasably inserted. The locking plug 24 can be releasably affixed to the collar 23 using a threaded grub fastener 25 or, alternatively, snap locked using a spring loaded pin sub-assembly (pin 21 and spring 22) affixed on the collar 23. An instrument guide insert 26 can be releasably placed inside the locking plug 24 to guide a surgical instrument (not shown) along a desired trajectory. Different diameters of the instrument guide insert 26 offer radial constraints for better positioning of the respective surgical instrument. Instrument guide inserts 26 of different lengths allow for precise guidance closer to a patient's body, as and when required by the instrument used.

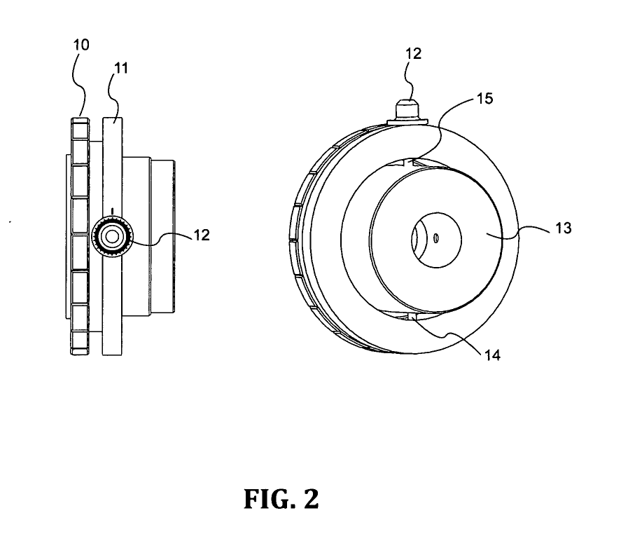

Referring to FIG. 2, for gimbal mechanisms, ring 11 comprises two axially aligned pins 14 and 15 that hold collar 13 in position while allowing it to rotate along the pin aligned axis. Pin 14 rests in a free hole between the gimbal mechanism ring 11 and collar 13, hence allowing collar 13 to rotate freely with respect to pin 14. Pin 15 can be rigidly connected to collar 13, passing through the gimbal mechanism ring 11 and is attached to a knob with a measuring scale 12. The knob with measuring scale 12 that is attached to pin 15 can be rotated to set the angle values for collar 13 to facilitate implantation through oblique trajectories.

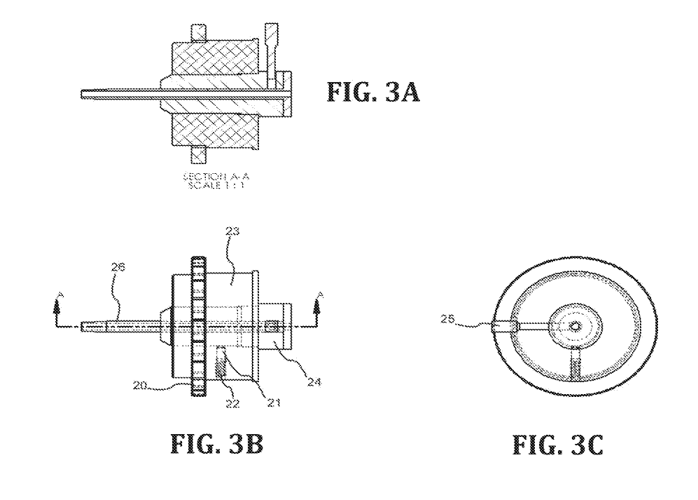

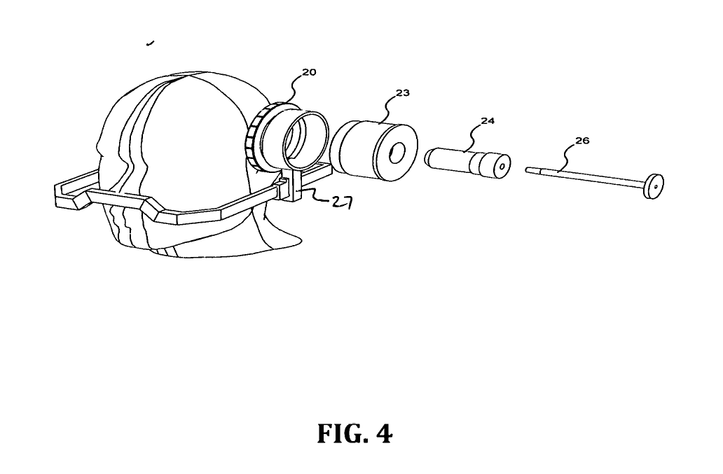

FIG. 3A depicts a cross-sectional view of an assembled Z-post adapter in accordance with an embodiment of the present disclosure; FIG. 3B depicts an elevation view of an assembled Z-post adapter in accordance with an embodiment of the present disclosure; and FIG. 3C depicts a plan view of an assembled Z-post adapter in accordance with an embodiment of the present disclosure. FIG. 4 depicts an exploded of a Z-post adapter with a stereotactic frame 27 in accordance with an embodiment of the present disclosure.

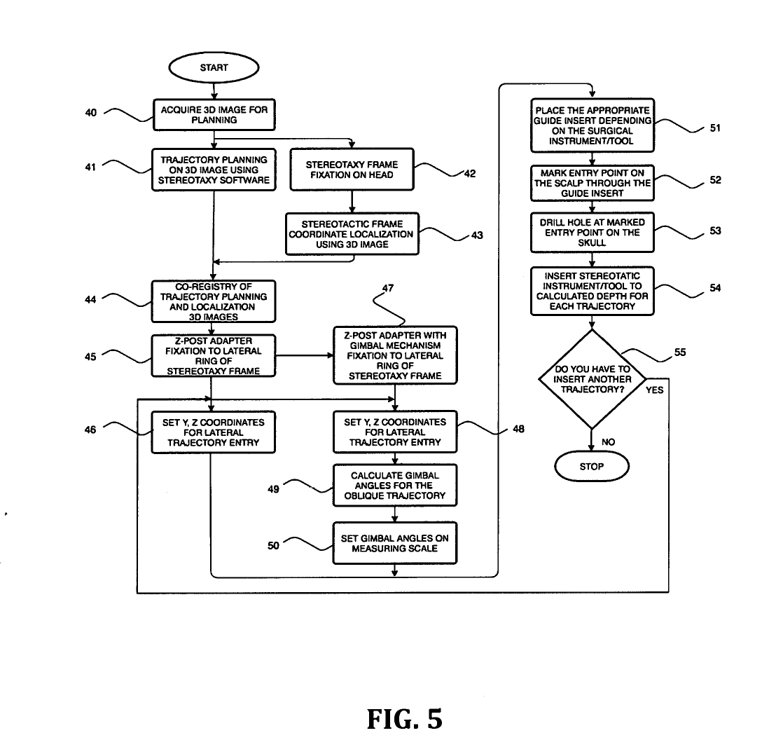

Referring now to FIG. 5, a flowchart presenting a use methodology of a Z-post adapter in accordance with an embodiment of the present disclosure is disclosed. In accordance with this embodiment:
1. Trajectories are planned using stereotaxy software applications that assign entry and target points based on conventional medical imaging (CT, MRI, etc.) 41.
2. The trajectory planning is done with standard reference to the AC-PC (anterior commissure-posterior commissure) plane.
3. For orthogonal trajectories, the trajectory is marked orthogonal to the sagittal plane. Oblique trajectories, when planned, will require the gimbal mechanism attachment to the device.
4. A stereotactic frame (Leksell ®, CRW ®, etc.) is fixed onto the patient's head using standard protocol 42, with care to ensure symmetric, aligned frame fixation.
5. The frame coordinate assignments are determined using a standard stereotaxy localizer box and volumetric imaging (standard frame stereotaxy protocol) 43.
6. The frame coordinate localizer 3-D image is coregistered with the trajectory planning 3-D image 44.
7. For orthogonal stereotaxy, the collar of the Z-post adapter is mounted on the ring of the stereotactic frame 45.
8. Coordinates y & z are set separately for the trajectory 46. The depth of insertion is calculated based on a device-specific formula utilizing the frame x value.
9. For oblique stereotactic trajectories, the Z-post adapter with gimbal mechanism is mounted on the ring of the stereotactic frame 47. The y & z coordinates are set separately for the trajectory 48 and gimbal angles are set on the measuring scale to reach the desired target through the planned trajectory 50. The gimbal angles will be calculated based on gimbal mechanism-specific formulae 49.
10. Place the appropriate guide insert into the locking plug 51, the entry point is marked on the scalp 52, an incision made and the skull is drilled in the trajectory under stereotactic guidance 53.
11. A stereotactic instrument or tool (for example, an SEEG electrode) is inserted to the calculated depth 54.
12. Steps are repeated for each trajectory, if required 55.

While the invention has been described in connection with preferred embodiments, it will be understood by those of ordinary skill in the art that other variations and modifications of the preferred embodiments described above may be made without departing from the scope of the invention. Other embodiments will be apparent to those of ordinary skill in the art from a consideration of the specification or practice of the invention disclosed herein.

What is claimed is:

1. A Z-post adapter, comprising:
a collar, forming an aperture, and adapted to releasably mount to a stereotactic frame;
a locking plug releasably coupled to said collar; and
an instrument guide releasably coupled to said locking plug.

2. The Z-post adapter of claim 1, further comprising:
a fastener to releasably couple said collar to said stereotactic frame.

3. The Z-post adapter of claim 1, further comprising:
a pin assembly to releasably snap lock said locking plug to said collar.

4. The Z-post adapter of claim 1, further comprising:
a threaded grub insert to releasably lock said locking plug to said collar.

5. The Z-post adapter of claim 1, further comprising:
an instrument guide to releasably couple to said locking plug.
